(19) United States
(12) Patent Application Publication
(10) Pub. No.: US 2012/0225106 A1
(43) Pub. Date: Sep. 6, 2012

(54) PERSONAL CARE COMPOSITIONS WITH SUSPENDED METAL OXIDES

(75) Inventors: Tracey Ross, Hewitt, NJ (US); Christine M. Barrett, Oakland, NJ (US)

(73) Assignee: ISP Investments Inc., Wilmington, DE (US)

(21) Appl. No.: 13/409,252

(22) Filed: Mar. 1, 2012

Related U.S. Application Data
(60) Provisional application No. 61/447,751, filed on Mar. 1, 2011.

(51) Int. Cl. A61K 8/81; A61K 8/92; A61Q 5/00; A61Q 17/04; A61Q 19/00; A61K 8/02; A61K 8/97

(52) U.S. Cl. 424/401; 424/59; 424/70.9

(57) ABSTRACT

Described herein are personal care compositions for use on the skin, hair, and lips that comprise: (A) at least one metal oxide, (B) lightly- to moderately-crosslinked PVP, and (C) at least one vehicle selected from the group consisting of: alcohols, esters, oils, glycols, and combinations thereof. The lightly- to moderately-crosslinked PVP suspends the metal oxide(s) whether or not water is added. Exemplary products embraced by the invention include sunscreens, diaper rash preparations, and anti-pruritic substances in the form of a lotion, cream, ointment, or gel.

### CROSS REFERENCE TO RELATED APPLICATION

The present application claims benefit of U.S. Provisional Application Ser. No. 61/447,751 filed on Mar. 1, 2011, the entire contents of which are hereby incorporated by reference.

### DESCRIPTION OF RELATED ART

The present invention relates to personal care compositions, and more specifically, to sun-care and skin-care compositions having one or more suspended metal oxides.

In the personal care arts many different types of formulated products exist to treat, protect, or prevent the user from the damaging effects of UV radiation and from bacterial, fungal, and/or yeast infections. Inevitably, these personal care compositions contain an aqueous or hydroalcoholic liquid vehicle with one or more metal oxides in combination with one or more co-ingredients that enhance protection, application, water/sweatproofness, and/or aesthetic qualities. The metals oxides include titanium dioxides, zinc oxides, and iron oxides. As a UV absorber, titanium oxides and zinc oxides are selected for their specific absorptive UV spectral range and their compatibility with the co-ingredients. Combinations of these UV absorbers often are needed to impart protection over the full UV spectrum. Zinc oxide is known to demonstrate broad UV spectrum protection, as well as anti-bacterial, anti-fungal, and anti-yeast activity. Calamine is a skin protectant/anti-itch product that contains both zinc oxide with iron oxide.

From a historical perspective, the sun- and skin-care compositions that are the subject of the present invention have been difficult to formulate, due in part to the relatively high densities of the metal oxides. To promote consistency, water-soluble rheology modifiers like xanthan gum, hydroxyethyl cellulose, and crosslinked poly(acrylic acid) may be the formulator's first choice. These water-based systems, however, may exhibit an undesirable texture or pilling (meaning forming balls) during application, such as to the skin. Hence, new and elegant compositions are needed to enhance the texture and application of these metal oxide-containing compositions.

Formulating these metal oxides is even more challenging when the composition contains an insufficient amount of water or even no water. Without adequate water to fully hydrate the available, known thickeners, the metal oxides tend to settle, causing product inhomogeneity. Yet, consumers often prefer alcohol-, oil-, ester-, or glycol-based compositions because they are fast-drying, non-greasy, non-whitening, and/or provide a pleasing skin-feel. Hence, a new thickener is needed that suspends metal oxides in sun- and skin-care compositions having an low or no water content.

Finally, metal oxides are also known to be incompatible with a number of thickeners such that phase separation and agglomeration result when they are combined. In this worse-case scenario the attempted composition can resemble cottage cheese or assume a ropey/stringy appearance. Hence, compositions also are needed that promote formulation compatibility without compromising product stability.

For example, Whitening Suncare Lotion—SPF 30+ (SU-0022 AP, 2007) a technical sheet by The Lubrizol Corp. describes a whitening suncare lotion using 4% titanium dioxide (“UV Blocker/Whitening Agent”) and 71% water. This second formula illustrates the fact that titanium dioxide may serve dual roles. Yet, regardless of its role, titanium dioxide remains difficult to formula due to settling and incompatibility in alcohol systems. Preferred sunscreen formulas contain at least 2% (w/w) (based on the total sunscreen weight) titanium dioxide. Similarly, Titanium Dioxide Sunscreen—SPF 25 Formula (SU-0008B, 2007), another publication by Lubrizol, teaches a titanium dioxide sunscreen that contains acrylates/C10-C30 alkyl acrylate crosspolymer (Carbopol® Ultrez 21) and 70% water. These formulas contain water because the crosspolymer is incompatible with the inorganic UV absorbers in alcohol systems.

One polymeric thickener recommended for suspending zinc oxide is the acrylates/acrylamide copolymer (and) mineral oil (and) polysorbate-85 product offered into commercial sale under the trade name Novemer™ by The Lubrizol Corp. The product brochure Novemer™* EC-1 Polymer (CP-30, 21 Jan. 2005) mentions this suspension behavior, but recommends a pH from 5.5 to 11. For pH from 5 to 6 the product bulletin recommends using Carbopol®, or instead to add mild acid. A related brochure, Sprayable After Sun Moisturizer (SU-0004) describes a sunscreen product using the Novemer™ product and zinc oxide, but in a water-based formula. Product scientists, however, require more robust formulation systems without limitations on pH for effectiveness.

All Lubrizol product information bulletins are hereby incorporated by reference in their entirety.

As it will be explained, the present invention is related to lightly- to moderately-crosslinked poly(N-vinyl-2-pyrrolidone) (PVP). This polymer was first introduced in U.S. Pat. No. 5,073,614. In that patent it is taught to be the precipitation polymerization product of N-vinyl-2-pyrrolidone monomer in an organic solvent, such as an aliphatic hydrocarbon solvent (preferably cyclohexane or heptane) or an aromatic hydrocarbon (such as toluene) in the presence of about 0.2% to 1% by weight of a crosslinking agent. The fine, white powders thus produced have an aqueous gel volume of about 15 mL to 150 mL of polymer, and a Brookfield viscosity in 5% aqueous solution of at least about 10,000 cP.

Lightly- to moderately-crosslinked PVP also was the subject of U.S. Pat. No. 5,139,770. Examples are provided in this patent for a cream rise (pH of 4), a hair conditioner (pH of 4), and a blow dry styling lotion (pH of 6), which have been pH-adjusted by the addition of citric acid or phosphoric acid.

U.S. Pat. No. 5,716,634 teaches a lightly-crosslinked N-vinyl lactam polymer in form of stable, clear, flowable, homogenized hydrogel, may be used as a carrier for cosmetic/pharma active for hair or skin use. Also, the production of lightly- to moderately-crosslinked PVP in an oil-in-water or water-in-oil emulsion is taught in U.S. Pat. No. 6,177,068.

Three PCT applications also disclose lightly- to moderately-crosslinked PVP in the personal care arts. WO 2010/105050 teaches substantially anhydrous, substantially non-alcoholic personal care compositions having the above-named polymer. WO 2010/105052 provides for compositions having at least: (a) one personal care acid at 0.5% addition level or more, or one pharmaceutical acid at 0.5% addition level or more, and (b) lightly- to moderately-crosslinked PVP. The third application, WO 2010/105030 claims composition comprising: (a) at least one active ingredient selected from the group consisting of an antiperspirant active and a deodorant active; and (b) a thickening agent, wherein the thickening agent comprises a strongly swellable, lightly to moderately crosslinked polyvinyl pyrrolidone.

Two other published applications relate to lightly -to moderately-crosslinked PVP. They are PCT publication numbers WO2011/137321, and WO2011/133511. The '321 publication, claims a two-part composition comprising at least two parts: (a) a first part having at least a hydroxide and (b) a second part having at least an alkaline material, wherein either said first part, or said second part, or both said first and said second parts comprise an effective amount of lightly- to moderately-crosslinked PVP. A preferred representative composition of the invention is a two-part hair relaxer having a pH greater than 11.

A summary of some properties of light- to moderately-crosslinked poly(N-vinyl-2-pyrrolidone) is given in Shih, J. S., “Characteristics of lightly crosslinked poly(N-vinylpyrrolidone),” Polymer Materials: Science & Engineering Preprint, 72, 374, 1995.

Still more information on this lightly crosslinked poly (N-vinyl-2-pyrrolidone) polymer is given in the following U.S. Pat. Nos. 5,162,417; 5,312,619; 5,622,168; 5,564,385; and 6,582,711.

The three U.S. patents ('614, '770, '634), the published PCT patent applications ('050, '052, '030, '321, '511), and the Shih article mentioned in the above paragraphs are hereby incorporated in their entirety by reference.

### SUMMARY OF THE INVENTION

Personal care compositions have been discovered that suspend metal oxides, extend formulation flexibility, and improve various application aesthetics, like the ease of application/spreadability, the lack of pilling, the speed of drying, a non-greasy feeling, and a non-whitening appearance. These compositions comprise at least one metal oxide, such a titanium dioxide, a zinc oxide, or an iron oxide, lightly- to moderately-crosslinked PVP, and at least one vehicle, such as an alcohol, ester, oil, or glycol. Not only is the metal oxide suspended throughout the composition without settling, but there is no phase separation or agglomeration. The invention provides the formation scientist with formulation flexibility, since the compositions are stable over a wide range of pH and temperature.

### DESCRIPTION OF PREFERRED EMBODIMENTS OF THE INVENTION

The present invention extends the sun- and skin-care formulation arts by offering compositions having suspended metal oxides without compromising product quality, uniformity, or performance. Examples of metal oxides include titanium dioxides, zinc oxides, and iron oxides, so that the sun- and skin-care formulas of the invention can protect the user from UV radiation, bacterial, fungal, and yeast infections (such as diaper rash), and from the irritation and itching of other skin conditions (such as contact dermatosis such as from poison ivy or sumac). Unlike other attempts, the present invention enables uniform and stable products that spread with a smooth, non-pilling elegance, and can dry quickly without a greasy or white appearance. The invention is particularly versatile, providing compositions having alcohol, ester, oil, or glycol vehicles. Because organic UV absorbers, like octocrylene and avobenzone, can be added, the invention's products can be crafted to protect the wearer from a broad spectrum of UV radiation. Similarly, the invention embraces a wide range of products, such as lotion sunscreens, sprayable sunscreens, diaper rash creams and lotions, moisturizers, creams, lip glosses/sticks, and products for hair care.

First, a few terms are defined to help frame the several preferred aspects of the invention.

The term lightly- to moderately-crosslinked PVP, unless otherwise noted, specifically refers to polymer essentially consisting of lightly- to moderately-crosslinked poly(N-vinyl-2-pyrrolidone) having at least one of the following characteristics: (1) an aqueous swelling parameter defined by its gel volume from about 15 mL/g to about 300 mL/g, more preferably from about 15 mL/g to about 250 mL/g, and most preferably from about 15 mL/g to about 150 mL/g, or (2) a Brookfield viscosity of 5% lightly- to moderately-crosslinked PVP in a liquid carrier comprising water at 25° C. of at least 2,000 cP, more preferably of at least about 5,000 cP, and most preferably of at least about 10,000 cP. Disclosure for these parameter ranges is provided in U.S. Pat. No. 5,073,614 and in Shih, J. S., et al. (1995). Synthesis methods for the lightly- to moderately-crosslinked PVP are disclosed in a number of references, including U.S. Pat. Nos. 5,073,614; 5,654,385; and 6,177,068. It is appreciated by a polymer scientist skilled in the art that the method of synthesis is immaterial, inasmuch as the produced polymer achieves at least one of the abovedefined parameters.

For example, U.S. patent '614 discloses different crosslinkers and crosslinker amounts that yield lightly- to moderately-crosslinked PVP suitable for the present invention. The effect of crosslinker amount on swell volume and viscosity is graphically presented in Shih, J. S., et al. (1995). Thus, the lightly- to moderately-crosslinked PVP may be produced by the precipitation polymerization method of the '614 patent, by the hydrogel method described in the '385 patent, or by the non-aqueous, heterogeneous polymerization method of the '068 patent. Certainly, other techniques are contemplated to synthesize this polymer, provided the product meets the aqueous swelling parameter and Brookfield viscosity requirements.

Final product viscosities may slightly vary for compositions containing lightly- to moderately-crosslinked PVP made by these different methods. Nonetheless, these variations are within the scope of the invention, as the lightly- to moderately-crosslinked PVPs thickens the color base and/or developer and/or color mix.

Unless otherwise specified, the term lightly- to moderately-crosslinked PVP does not refer to swellable but water-insoluble crosslinked PVP, such as the type sold into commercial trade under the trade name Polyclar® by International Specialty Products, which differs from the abovedescribed lightly- to moderately-crosslinked PVP.

The term viscosity refers to the proportionality coefficient between shear stress and shear rate, and describes a composition's resistance to flow. Because viscosity is dependent on shear rate, specific measurement information (such as viscometer, flow apparatus/spindle, and shear rate) is required to properly define viscosity. As used herein, viscosity refers to the proportionality coefficient determined from low shear rate, rotational flow, especially the viscosity measured by the Brookfield LVT and Brookfield RVT viscometers operating at 10 revolutions per minute (rpm) at 25° C. References describing the Brookfield measurement of viscosities include the following, each of which is hereby incorporated in its entirety by reference: Thibodeau, L., “Measuring viscosity of pastes,” American Laboratory News, June 2004; McGregor, R. G., “Shelf life: does viscosity matter?” Pharmaceutical Online, Oct. 31, 2007; and McGregor, R. G., “When ointments disappoint, the viscosity story,” Brookfield Engineering brochure.

The terms ultraviolet and UV mean electromagnetic radiation, especially solar electromagnetic radiation, with a wavelength from about 100 nm to about 400 nm, and includes the UV-A, UV-B, and UV-C subclassifications of such radiation.

The term UV-A means ultraviolet electromagnetic radiation with a wavelength from about 320 nm to about 400 nm, and includes UV-A1 (from about 340 nm to about 400 nm) and UV-A2 (from about 320 nm to about 340 nm).

The term UV-B means ultraviolet electromagnetic radiation with a wavelength from about 290 nm to about 320 nm.

The term UV-C means ultraviolet electromagnetic radiation with a wavelength from about 200 nm to about 290 nm.

The term UV absorber means any entity that absorbs, scatters, and/or reflects UV radiation.

The term personal care compositions refers to sun-care compositions and skin-care compositions.

The term sun-care compositions refers to personal care and/or pharmaceutical compositions comprising an effective amount of UV-absorbing compositions, including the ultraviolet-absorbing compounds of this invention. Sun-care compositions include beach and non-beach products that are applied to the face, décolleté, lips, hands, and to skin in general to treat and/or protect against erythema, burns, wrinkles, lentigo (“liver spots”), skin cancers, keratotic lesions, and cellular changes of the skin; and to hair to treat and/or protect against color changes, lack of luster, tangles, split ends, unmanageability, and embrittlement.

The term skin-care compositions refers to personal care and/or pharmaceutical compositions used in the treatment or prevention of skin conditions, such as primary or secondary bacterial, fungal infections, such as those caused by Candida albican, Pseudomonas aeruginosa, and Staphylococcus aureus. (Other infective agents can be identified by one skilled in the art.) These skin infections commonly occur in diaper rash. The skin-care compositions also can be used to treat contact dermatoses, like those that occur from various rash-producing plants (e.g., poison ivy) or insect bites/stings.

All percentages, ratio, and proportions used herein are based on a weight basis unless other specified.

Surprisingly, compositions have been discovered that suspend metal oxides without phase separation or instability, and instead offer an elegant presentation of sun- and skin-care compositions. Unlike other rheology modifiers, pH modification to not required for clarity or thickening, as these properties are invariant with lightly- to moderately-crosslinked PVP. The compositions comprise at a minimum: (A) at least one metal oxide, (B) lightly- to moderately-crosslinked PVP, and (C) at least one vehicle selected from the group consisting of: alcohols, esters, oils, glycols, and combinations thereof. Other ingredients may be included in the compositions, and are discussed in detail after the following information on the essential components.

### Metal Oxide

The invention requires at least one metal oxide, since this family of materials has proved difficult to formulate without settling, product inhomogeneity, and/or phase separation. The term metal oxide encompasses titanium dioxides, zinc oxides, and iron oxides. These materials can perform a number of roles in the sun- and skin-care compositions, including absorbing UV radiation, exhibiting anti-bacterial, anti-fungal, and/or anti-yeast activity, and/or as an anti-pruritic for sunburn, insect bites/stings, or contact dermatosis. Micronized forms of these metal oxides are currently available that enhanced properties without imparting the traditional opaqueness that may be deemed aesthetically unappealing. Furthermore, these metal oxides are marketed in a variety of particle sizes, coatings (if coated), dispersions, and suspensions and find wide application in the personal care and pharmaceutical arts.

Examples of titanium dioxides and zinc oxides suitable for use in the present invention include, without limitation, the materials disclosed in: U.S. Pat. Nos. 4,246,040; 4,581,293; 5,068,056; 5,573,753; 5,599,529; 5,891,237; 5,914,101; 6,267,949; 6,683,130; 6,855,311; 7,220,305; 7,503,970; U.S. patent application 2009/0324657; international applications WO 2010/068687, WO 2009/126722, and WO 2008/067186; and G. B. patents 1,408,425; 2,205,088; and 2,206,339. Each patent and patent application is hereby incorporated in its entirety by reference.

Brand name titanium dioxide products offered for commercial include the following: A10-TiO2-11S7, A10-TiO2-MS7, A10-TiO2-TTS7, TTO-V-4, JTTO-MS7, TTO-S-4, TTO-TTB7, MT-500B-NJE5, MT-600B-11S5, MT-600B-MS7, PF-7 TiO2 MT-600B, PF-10 SST-65C-S, TiO2 STT-65S-13, MPT-139, TiO2 KQ-14, TiO2 KQ-MS4, STN-405, UV-CAPSULE60, EA-209/MT-100T, STZAS-504, DAIAMID MSP-TiO50, MPT-154-NJE8, TEL-100-NJE5, TiO₂ TEL-100, TTO-NJE8, TNP45TEL, EMP50TEL, ISDMP50TEL, INTNP50TEL, and GCP55TEL products by Kobo Products Inc. (South Plainfield, N.J.)

Brand name zinc oxide products include: the Zano® 10 product line and the Xperse® product lines by Umicore (Brussels, BE); Zinc Oxide Neutral, Zinc Oxide NDM, and Zinc Oxide PI all by Symrise (Holzminden, Del.); nanosized zinc oxide by Sunjin Chemical Company (Busan, KR); Sunsafe-Znom by UniProma (Jiangsu, CN), ZnO-C, ZnO—C12, Zno-C-NJE3, Zno-C-DMC2, GC70MZCJ, CMX80MZCM, DM2X80MZCM, MTMX80MZCM, STZAS-504, DAIAMID MSP-ZnO50, HMZ-50 and JOP80MZCJ products by Kobo Products Inc. (South Plainfield, N.J.).

Zinc oxide demonstrates antimicrobial, antifungal and skin protectant properties (Mitchnick, M. A., et al., “Microfine zinc oxide (Z-Cote) as a photostable UVA/UVB sunblock agent,” J Amer Acad Derm, 40, 1, 85-90; which is incorporated in its entirety by reference), and is the only sunscreen ingredient to appear on more than one FDA monograph. Zinc oxide is the number one active ingredient recommended by pediatricians for the treatment of diaper rash.

Like the other metal oxides named above, iron oxides have a long history of use in personal care and pharmaceutical compositions. A number of iron oxides exist, due to the These oxides include: iron (II) oxide (FeO), iron (II, III) oxide (Fe₃O₄), and iron (III) oxide (Fe₂O₃). The latter oxide, Fe₂O₃, is especially preferred in the present invention, as it finds widespread use along with zinc oxide in calamine lotions. Iron (III) oxide occurs in four commercially-important morphologies: α-, β-, γ-, and ε-iron (III) oxides.

Personal care compositions often contain the various combinations of two or more of titanium dioxide(s), zinc oxide(s), and iron oxide(s) to contribute to the final product color and/or opacity. An example of one such combination is international patent application WO 2008/042326, which is hereby incorporated in its entirety by reference. Indeed, It will be appreciated, however, that regardless of its function, these oxides present the formulator with challenges noted earlier.

The metal oxide addition level can assume wide ranges, since it is recognized that they can serve as a UV absorber, an anti-pruritic, colorant (typically white or red/orange), and/or an anti-bacterial/anti-fungal/anti-yeast agent. Regardless of the functional role, apart from the present invention the metal oxide is problematic to suspend and formulate. Typically, the addition level ranges from 0.1% (w/w) to 40% (w/w) based on the total weight of the personal-care composition.

When the metal oxide functions (at least in part) as a UV absorber, the metal oxide(s) is generally added at 2% (w/w) or more. The invention embraces addition levels less than 2% especially when the metal oxide is used in combination with organic UV absorbers. A review of commercial sunscreen formulations shows that addition levels greater than 2% are common. Some products contain 7% (w/w) or more of one or more titanium dioxide and/or zinc oxide.

Commercial diaper rash formulas typically contain more zinc oxide than found in sun-care products. For example, commercial diaper rash preparations containing 10% (w/w), 15% (w/w), and even 40% (w/w) zinc oxide are available to the consumer.

### Lightly- to Moderately Crosslinked PVP

Compositions of the invention also comprise an effective amount of lightly- to moderately-crosslinked PVP. The term “effective” is taken to make an amount to provide sufficient rheology to suspend the metal oxide(s) and meet the required product performance specifications. Understandably, sun- and skin-care compositions having a low viscosity, such as a sprayable sunscreen or sunblock, may require less lightly- to moderately-crosslinked PVP than an ointment, cream, wax or paste-like product, such as a lipstick, hair gel/wax/paste, diaper rash cream, or stick sun-block.

Typically, an effective amount of this polymer ranges from 0.5% (w/w) to 20% (w/w), and more typically the addition level ranges from 1% (w/w) to 10% (w/w). An example of the invention illustrates a lotion sun-care composition having 3% (w/w) usage level of lightly- to moderately-crosslinked PVP.

### Vehicle

Naturally, compositions of the invention require one or more vehicles to help distribute, disperse, dilute, and/or present the compositions for their various uses. The vehicle can be one of several types of materials commonly used in sun- and skin-care compositions, such as alcohols, esters, oils, and glycols. Combinations of these vehicles can be used. Additionally, disclosure of non-aqueous compositions with lightly- to moderately-crosslinked PVP is made in pending U.S. patent application

Alcohols are those vehicles having at least one hydroxyl group. Preferred alcohols include ethanol, 1-propanol, 2-propanol, and their combinations.

Esters are those vehicles having at least one —C(O)O— moiety. Many different esters are known in the cosmetic and personal care arts. Exemplary esters include, without limitation, the butyl ester of PVM/MA copolymer, cetyl ethylhexanoate, decyl oleate, diisopropyl adipate, diisopropyl dimer dilinoleate, diisostearyl maleate, dioctyl maleate, ethylhexyl palmitate, 2-ethylhexyl-12-hydroxystearate, ethyl ester of PVM/MA copolymer, isocetyl stearate, isocetyl stearoyl stearate, isodecyl neopentanoate, isodecyl oleate, isopropyl ester of PVM/MA copolymer, isostearyl neopentanoate, myristyl laurate, myristyl myristate, neopentyl glycol diethylhexanoate, octyldodecyl stearate, phenethyl benzoate, triisostearyoyl polyglyceryl-3 dimer dilinoleate, and combinations thereof.

Useful oils include those oils from plant, animal, and synthetic origins, such as sunflower oil, corn oil, soy oil, avocado oil, jojoba oil, squash oil, raisin seed oil, sesame seed oil, walnut oils oil, glycerol tricaprocaprylate, Purcellin oil, jojoba oil, eucalyptus oil, lavender oil, vetiver oil, litsea cubeba oil, lemon oil, sandalwood oil, rosemary oil, chamomile oil, savory oil, nutmeg oil, cinnamon oil, hyssop oil, caraway oil, orange oil, geranium oil, cade oil, oil of bergamot, hexadecane, oil of paraffin, and combinations thereof.

Synthetic oils also are known and include the following materials: polyolefins such as polybutene, polyisobutene, and polydecenes. Also suitable are the various silicone oils, being polymerized siloxanes that are the silicon-based analogues of carbon-based compounds. This concept of silicone oils includes polyorganosiloxanes, being siloxanes with one or more organic chains. Examples of silicone oils are, without limitation, C24-C28 alkyl methicone (CAS no. 158061-44-0), C30-C45 alkyl methicone (CAS no. 246864-88-0), the many compounds having “dimethicone” in their INCI name (e.g., cetyl dimethicone, cetyl dimethicone copolyol, dimethicone bis-aminohydroxypropyl copolyol, dimethicone copolyol), the many compounds having “dimethiconol” in their INCI name (e.g., dimethiconol, dimethiconol beeswax, dimethiconol behenate), the many compounds having “methylsilanol” in their INCI name (e.g., methylsilanol carboxymethyl theophylline alginate, methylsilanol elastinate, methylsilanol spirulinate), polysilicones 1 through 11, and silicone quaterniums 1 through 13. Combinations of these oils may be used.

Useful glycols include, but are not limited to, propylene glycol, butylene glycol, diethylene glycol, dipropylene glycol, polyethylene glycol, glycerin, and mixtures thereof.

Many more examples and descriptions of suitable titanium dioxides, zinc oxides, vehicles, and optional can be found in the following references, each of which is herein incorporated in its entirety by reference: “Inventory and common nomenclature of ingredients employed in cosmetic products,” Official Journal of the European Union, 5.4.2006, pages L 97/1 through L 97/528; and International Cosmetic Ingredient Dictionary and Handbook, 13^{th} edition, ISBN: 1882621476, published by The Personal Care Products Council in January 2010.

Typically, one or more vehicles are added from between 5% (w/w) to 95% (w/w), depending on the desired product characteristics, mode of delivery, and viscosity.

### Optional Ingredients

Given the nature of the sun- and skin-care compositions described by the present invention, it is understandable that additional ingredients may be formulated with the three essential ingredients without altering the scope of the current invention. These optional ingredients may be added for any number of reasons, for example to help create aesthetically-pleasing product forms, assist delivery/presentation of the product, promote stability, provide color or fragrance, modulate the rheology, and/or deliver active ingredients such as antioxidants, antibiotics, moisturizers, or anti-wrinkle agents.

Examples of these optional ingredient are liquid carriers, waxes, surfactants, emulsifiers, rheology modifiers, lubricants, diluents, humectants, antioxidants, preservatives, antibiotics, and viscosity and clarity synergists. antiradical agents, antioxidants, vitamins and pro-vitamins, fixing agents, oxidizing agents, reducing agents, dyes, cleansing agents, anionic, cationic, nonionic and amphoteric surfactants, thickeners, perfumes, pearlizing agents, stabilizers, pH adjusters, filters, preservatives, cationic and nonionic polyether associative polyurethanes, vegetable oils, mineral oils, synthetic oils, polyols such as glycols and glycerol, silicones, aliphatic alcohols, colorants, bleaching agents, highlighting agents and sequestrants. These additives are present in the composition in proportions that may range from 0% to 50% by weight in relation to the total weight of the composition. The precise amount of each additive may be determined by an expert in the field according to its nature and its function.

Optional ingredients also include viscosity and clarity synergists as discussed in U.S. provisional patent application 61/325,673.

### Optional Ingredient: Water

In preferred embodiments of the invention, the sun- and skin-care compositions further comprise water in addition to at least one metal oxide, lightly- to moderately-crosslinked PVP, and vehicle.

As described above, the prior art shows that metal oxides cannot be formulated in anhydrous compositions without encountering phase separation or ingredient incompatibility. Similarly, these metal oxides cannot be formulated in systems having a small amount of water, but less than needed to fully dissolve the thickener (e.g., xanthan gum, hydroxyethyl cellulose, Carbopol®).

In an embodiment of the present invention, a small amount of water is added to one or more alcohols, esters, oils, or glycols. In this context, a “small amount” of water means that a thickened and suspended metal oxide composition cannot be made when the lightly- to moderately-crosslinked PVP is replaced by a traditional polymer, such as hydroxyethylcellulose, guar gum, xanthan gum, or a crosslinked poly(acrylic acid). Generally speaking this requirement is met when water is added from between 0% (i.e., anhydrous) to 15% (w/w) of the total formula. Note that this embodiment of the invention does not provide for a water-only vehicle, but rather for alcohol/water, ester/water, oil/water, and glycol/water systems.

This option of the invention provides additional formulation flexibility, as it can help to reduce the volatile organic compound (VOC) content of the compositions, allowing end-user products that are “greener” for the environment. The formulation scientist can balance the amounts of vehicle (i.e., alcohol, oil, glycol, and/or ester) and water to optimize user perceptions of product spreadability, texture/skin feel, and evaporation.

### Optional Ingredient: Other UV Absorbers

It may be preferred to formulate the personal care compositions with one or more additional UV absorbers. The addition of these UV absorbers may provide any number of benefits, include (without limitation): extended the range of UV spectrum protection, provide a customized range of UV spectrum protection (e.g., ranges of UV-A and/or UV-B absorption), or stabilize labile UV absorbers. Examples of photolabile UV absorber include, without limitation: avobenzone, para-aminobenzoic acid (PABA) derivatives, cinnamates, and dibenzoyl methane derivatives, all of which degrade over time and reduce UV protection. A description of UV absorbers is provided in The Encyclopedia of Ultraviolet Filters (ISBN: 978-1-932633-25-2) published by Allured Publishing Corp., the contents of which are incorporated herein its entirety by reference.

UV absorbers that may be formulated with the ultraviolet-absorbing compounds include: octyl salicylate (2-ethylhexyl salicylate, Escalol® 587); pentyl dimethyl PABA; octyl dimethyl PABA (padimate 0, Escalol® 507); benzophenone-1; benzophenone-6 (Uvinul® D-49); 2-(2H-benzotriazole-2-yl)-4,6-di-tert-pentylphenol (Uvinul® 3028); ethyl-2-cyano-3,3-diphenylacrylate (Uvinul® 3035); homomethyl salicylate (homosalate); bis-ethylhexyloxyphenol methoxyphenyl triazine (bemotrizinol, Tinosorb® S); methyl-(1,2,2,6,6-pentamethyl-4-piperidyl)-sebacate (Uvinul® 4092H); benzenepropanoic acid, 3,5-bis(1,1-dimethyl-ethyl)-4-hydroxy-, C7-C9 branched alkyl esters (Irganox® 1135); 2-(2H-benzotriazole-2-yl)-4-methylphenol (Uvinul® 3033P); diethylhexyl butamido triazone (iscotrizinol); amyl dimethyl PABA (lisadimate, glyceryl PABA); 4,6-bis(octylthiomethyl)-o-cresol (Irganox® 1520); CAS number 65447-77-0 (Uvinul® 5062H, Uvinul® 5062GR); red petroleum; ethylhexyl triazone (Uvinul® T-150); octocrylene (Escalol® 597); isoamyl-p-methoxycinnamate (amiloxate, Neo Heliopan® E1000); drometrizole; titanium dioxide; 2,4-di-tert-butyl-6-(5-chloro-2H-benzotriazole-2-yl)-phenol (Uvinul® 3027); 2-hydroxy-4-octyloxybenzophenone (Uvinul® 3008); benzophenone-2 (Uvinul® D-50); diisopropyl methylcinnamate; PEG-25 PABA; 2-(1,1-dimethylethyl)-6-[[3-(1,1-demethylethyl)-2-hydroxy-5-methylphenyl]methyl-4-methylphenyl acrylate (Irganox® 3052); drometrizole trisiloxane (Mexoryl® XL); menthyl anthranilate (meradimate); bis-(1,2,2,6,6-pentamethyl-4-piperidyl)-sebacate; butyl methoxydibenzoylmethane (avobenzone, Escalol® 517); 2-ethoxyethyl p-methoxycinnamate (cinnoxate); benzylidene camphor sulfonic acid (Mexoryl® SL); dimethoxyphenyl-[1-(3,4)]-4,4-dimethyl 1,3-pentanedione; zinc oxide; N,N′-hexane-1,6-diyl-bis[3-(3,5-di-tert-butyl-4-hydroxyphenylpropionamide)] (Irganox® 1098); pentaerythritol tetrakis[3-(3,5-di-tert-butyl-4-hydroxyphenyl)propionate] (Irganox® 1010); 2,6-di-tert-butyl-4-[4,6-bis(octylthio)-1,3,5-triaziN-2-ylamino]phenol (Irganox® 565); 2-(2H-benzotriazole-2-yl)-4,6-bis(1-methyl-1-phenylethyl)phenol (Uvinul® 3034); trolamine salicylate (triethanolamine salicylate); diethylanolamine p-methoxycinnamate (DEA methoxycinnamate); polysilicone-15 (Parsol® SLX); CAS number 152261-33-1 (Uvinul® 5050H); 4-methylbenzylidene camphor (Eusolex® 6300, Parsol® 5000); bisoctrizole (Tinosorb® M); benzenamine, N-phenyl-, reaction products with 2,4,4-trimethylpentene (Irganox® 50507); sulisobenzone, Escalol® 577); (2-ethylhexyl)-2-cyano-3,3-diphenylacrylate (Uvinul® 3039); digalloyl trioleate; polyacrylamido methylbenzylidene camphor; glyceryl ethylhexanoate dimethoxycinnamate; 1,3-bis-[(2′-cyano-3′,3′-diphenylacryloyl)oxy]-2,2-bis-{[(2′-cyano-; bis-(2,2,6,6-tetramethyl-4-piperidyl)-sebacate (Uvinul® 4077H); benzophenone-5; 1,3,5-tris(3,5-di-tert-butyl-4-hydroxybenzyl)-1,3,5-triazine-2,4,6(1H,3H,5H)-trione (Irganox® 3114); hexamethylendiamine (Uvinul® 4050H); benzophenone-8 (dioxybenzone); ethyl-4-bis(hydroxypropyl)aminobenzoate (roxadimate); 6-tert-butyl-2-(5-chloro-2H-benzotriazole-2-yl)-4-methylphenol (Uvinul® 3026); p-aminobenzoic acid (PABA); 3,3′,3″,5,5′,5″-hexa-tert-butyl-α-α′-α″-(mesitylene-2,4,6-triyl)tri-p-cresol (Irganox® 1130); lawsone with dihydroxyacetone; benzophenone-9 (Uvinul® DS-49); benzophenone-4; ethylhexyl dimethoxy benzylidene dioxoimidazoline propionate; N,N′-bisformyl-N,N′-bis-(2,2,6,6-tetramethyl-4-piperidinyl)-; 3-benzylidene camphor (Mexoryl® SD); terephthalylidene dicamphor sulfonic acid; camphor benzalkonium methosulfate (Mexoryl® SO); bisdisulizole disodium (Neo Heliopan® AP); etocrylene; ferulic acid; 2-(2H-benzotriazole-2-yl)-4-(1,1,3,3-tetramethylbutyl)-phenol (Uvinul® 3029); 4,6-bis(dodecylthiomethyl)-o-cresol (Irganox® 1726); beta-2-glucopyranoxy propyl hydroxy benzophenone; phenylbenzimidazole sulfonic acid (ensulizole, Eusolex® 232, Parsol® HS); benzophenone-3 (oxybenzone, Escalol® 567); diethylamine hydroxybenzoyl hexylbenzoate (Uvinul® A Plus); 3′,3′-diphenylacryloyl)oxy]methyl}-propane (Uvinul® 3030); and ethylhexyl p-methoxycinnamate (Escalol® 557).

It is recognized that the availability of UV absorbers in sun-care compositions often depends on local regulatory laws; hence, the above list may include UV absorbers that are not allowed in certain regions.

Preferred are those sun care composition having one or more UV absorber selected from the following: p-aminobenzoic acid (PABA), Padimate O, ensulizole, cinoxate, benzophenone-3, enzophenone-8, homosalate, meradimate, octocrylene, 2-ethylhexyl-p-methoxycinnamate, octyl salicylate, sulisobenzone, trolamine salicylate, avobenzone, ecamsule, titanium dioxide, zinc oxide, 4-methylbenzylidene, Tinosorb® M, neo heliopan AP, mexoryl XL, benzophenone-9, Uvinul® T150, Uvinul® A Plus, Uvasorb® HEB, Parsol® SLX, and isopentenyl-4-methoxycinnamate.

### Optional Ingredients: Surfactants

Surfactants also display a synergistic interaction with lightly- to moderately-crosslinked PVP to increase viscosity. Surfactants suitable for use in the present invention include those selected from the anionic, cationic, amphoteric (also called zwitterionic), and non-ionic families of surfactants, and blends thereof.

Anionic surfactants include alkyl sulfate, alkyl ethoxylated sulfate, and mixtures thereof. These materials have the respective formula (1) ROSO₃M and (2) RO(C₂H₄O)xSO₃M, wherein R is alkyl or alkenyl of from about 8 to about 30 carbon atoms, x is 1 to 10, and M is H or a salt-forming cation such as ammonium, alkanolamine containing C1-C3 alkyl groups such as triethanolamine, and monovalent and polyvalent metals such as the alkaline and alkaline earth metals. Preferred metals include sodium, potassium, magnesium, and calcium. The cation M, of the anionic surfactant should preferably be chosen such that the anionic surfactant component is water soluble. Solubility of anionic surfactants, in general, will depend upon the particular anionic surfactants and cations chosen. It is preferred that the anionic surfactant be soluble in the composition hereof.

Preferably, R has from about 10 to about 18 carbon atoms in both the alkyl and alkyl ethoxylated sulfates. The alkyl ethoxylated sulfates are typically made as condensation products of ethylene oxide and monohydric alcohols having from about 8 to about 24 carbon atoms. The alcohols can be derived from fats, e.g., coconut oil, palm kernel oil, or tallow, or can be synthetic. Such alcohols are preferably reacted with about 1 to about 10, more preferably from about 1 to about 4, most preferably from about 2 to about 3.5, molar proportions of ethylene oxide and the resulting mixture of molecular species having, for example, an average of 3 moles of ethylene oxide per mole of alcohol, is sulfated and neutralized.

Specific examples of alkyl ether sulfates which may be used in the present invention are sodium and ammonium salts of coconut alkyl triethylene glycol ether sulfate; tallow alkyl triethylene glycol ether sulfate, and tallow alkyl hexaoxyethylene sulfate. Highly preferred alkyl ether sulfates are those comprising a mixture of individual compounds, said mixture having an average alkyl chain length of from about 12 to about 16 carbon atoms and an average degree of ethoxylation of from about 1 to about 4 moles of ethylene oxide. The sulfate surfactant is preferably comprised of a combination of ethoxylated and nonethoxylated sulfates. Alkyl sulfates can provide excellent cleaning and lather performance. Alkyl ethoxylated sulfates can provide excellent cleaning performance.

Other suitable anionic detersive surfactants include, but are not limited to water-soluble salts of organic, sulfuric acid reaction products of the general formula R₁SO₃M where R₁ is selected from the group consisting of a straight or branched chain, saturated aliphatic hydrocarbon radical having from about 8 to about 24, preferably about 10 to about 18, carbon atoms; and M is a cation such as ammonium, alkanolamines, such as triethanolamine, monovalent metals, such as sodium and potassium, and polyvalent metal cations, such as magnesium, and calcium. The cation M, of the anionic detersive surfactant should be chosen such that the detersive surfactant component is water soluble. Solubility will depend upon the particular anionic detersive surfactants and cations chosen. Examples of such detersive surfactants are the salts of an organic sulfuric acid reaction product of a hydrocarbon of the methane series, including iso and n-paraffins, having about 8 to about 24 carbon atoms, preferably about 10 to about 18 carbon atoms and a sulfonating agent, e.g., SO₃, H₂SO₄, obtained according to known sulfonation methods, including bleaching and hydrolysis. Preferred are alkali metal and ammonium sulfonated C10-C18 n-paraffins.

Suitable classes of nonionic surfactants also include, but are not limited to:
1. The polyethylene oxide condensates of alkyl phenols, e.g., the condensation products of alkyl phenols having an alkyl group containing from about 6 to about 12 carbon atoms in either a straight chain or branched chain configuration, with ethylene oxide, the said ethylene oxide being present in amounts equal to from about 10 to about 60 moles of ethylene oxide per mole of alkyl phenol. The alkyl substituent in such compounds may be derived from polymerized propylene, diisobutylene, octane, or nonane, for example.
2. Those derived from the condensation of ethylene oxide with the product resulting from the reaction of propylene oxide and ethylene diamine products which may be varied in composition depending upon the balance between the hydrophobic and hydrophilic elements which is desired. For example, compounds containing from about 40% to about 80% polyoxyethylene by weight and having a molecular weight of from about 5,000 to about 11,000 resulting from the reaction of ethylene oxide groups with a hydrophobic base constituted of the reaction product of ethylene diamine and excess propylene oxide, said base having a molecular weight of the order of about 2,500 to about 3,000, are satisfactory.
3. The condensation product of aliphatic alcohols having from about 8 to about 18 carbon atoms, in either straight chain or branched chain configuration, with ethylene oxide, e.g., a coconut alcohol ethylene oxide condensate having from about 10 to about 30 moles of ethylene oxide per mole of coconut alcohol, the coconut alcohol fraction having from about 10 to about 14 carbon atoms.
4. Long chain tertiary amine oxides corresponding to the following general formula: R₁R₂R₃N→O, wherein R₁ contains an alkyl, alkenyl or monohydroxy alkyl radical of from about 8 to about 18 carbon atoms, from 0 to about 10 ethylene oxide moieties, and from 0 to about 1 glyceryl moiety, and R₂ and R₃ contain from about 1 to about 3 carbon atoms and from 0 to about 1 hydroxy group, e.g., methyl, ethyl, propyl, hydroxyethyl, or hydroxypropyl radicals. The arrow in the formula is a conventional representation of a semipolar bond. Non-limiting examples of amine oxides suitable for use in this invention include dimethyl-dodecylamine oxide, dimethyloctylamine oxide, dimethyl-decylamine oxide, dimethyl-tetradecylamine oxide, 3,6,9-tri-oxaheptadecyldiethylamine oxide, di(2-hydroxyethyl)-tetradecylamine oxide, 2-dodecoxyethyldimethylamine oxide, 3-dodecoxy-2-hydroxypropyldi(3-hydroxypropyl)amine oxide, dimethylhexadecylamine oxide.
5. Long chain tertiary phosphine oxides corresponding to the following general formula: RR′R″P→O wherein R contains an alkyl, alkenyl or monohydroxyalkyl radical ranging from about 8 to about 18 carbon atoms in chain length, from 0 to about 10 ethylene oxide moieties and from 0 to about 1 glyceryl moiety and R′ and R″ are each alkyl or monohydroxyalkyl groups containing from about 1 to about 3 carbon atoms. The arrow in the formula is a conventional representation of a semipolar bond. Examples of suitable phosphine oxides include, but are not limited to: dodecyldimethylphosphine oxide, tetradecyldimethylphosphine oxide, tetradecylmethylethylphosphine oxide, 3,6,9,-trioxaoctadecyidimethylphosphine oxide, cetyidimethylphosphine oxide, 3-dodecoxy-2-hydroxypropyldi(2-hydroxyethyl)phosphine oxide, stearyldimethylphosphine oxide, cetylethylpropylphosphine oxide, oleyldiethylphosphine oxide, dodecyldiethylphosphine oxide, tetradecyldiethylphosphine oxide, dodecyldipropylphosphine oxide, dodecyldi(hydroxymethyl)phosphine oxide, dodecyldi(2-hydroxyethyl)phosphine oxide, tetradecylmethyl-2-hydroxypropylpho sphine oxide, oleydimethylphosphine oxide, 2-hydroxydodecyldimethylphosphine oxide.
6. Long chain dialkyl sulfoxides containing one short chain alkyl or hydroxy alkyl radical of from about 1 to about 3 carbon atoms (usually methyl) and one long hydrophobic chain which include alkyl, alkenyl, hydroxy alkyl, or keto alkyl radicals containing from about 8 to about 20 carbon atoms, from 0 to about 10 ethylene oxide moieties and from 0 to about 1 glyceryl moiety. Examples include, but are not limited to: octadecyl methyl sulfoxide, 2-ketotridecyl methyl sulfoxide, 3,6,9,-trixaoctadecyl 2-hydroxyethyl sulfoxide, dodecyl methyl sulfoxide, oleyl 3-hydroxypropyl sulfoxide, tetradecyl methyl sulfoxide, 3-methoxytridecyl methyl sulfoxide, 3-hydroxytridecyl methyl sulfoxide, 3-hydroxy-4-dodecoxybutyl methyl sulfoxide.
7. Polyalkylene oxide modified dimethylpolysiloxanes, also known as dimethicone copolyols. These materials include the polyalkylene oxide modified dimethylpolysiloxanes of the following formulae:

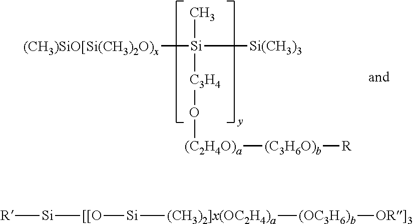

wherein R is hydrogen, an alkyl group having from 1 to about 12 carbon atoms, an alkoxy group having from 1 to about 6 carbon atoms or a hydroxyl group; R′ and R″ are alkyl groups having from 1 to about 12 carbon atoms; x is an integer of from 1 to 100, preferably from 20 to 30; y is an integer of 1 to 20, preferably from 2 to 10; and a and b are integers of from 0 to 50, preferably from 20 to 30. Dimethicone copolyols among those useful herein are disclosed in the following patent documents: U.S. Pat. No. 4,122,029; U.S. Pat. No. 4,265,878; and U.S. Pat. No. 4,421,769. Commercially available dimethicone copolyols, useful herein, include Silwet Surface Active Copolymers (manufactured by the Union Carbide Corporation); Dow Corning Silicone Surfactants (manufactured by the Dow Corning Corporation); Silicone Copolymer F-754 (manufactured by SWS Silicones Corp.); and Rhodorsil 70646 Fluid (manufactured by Rhone Poulenc, Inc.).

Specifically, anionic surfactants for use in the invention include: ammonium lauryl sulfate, ammonium laureth sulfate, triethylamine lauryl sulfate, triethylamine laureth sulfate, triethanolamine lauryl sulfate, triethanolamine laureth sulfate, monoethanolamine lauryl sulfate, monoethanolamine laureth sulfate, diethanolamine lauryl sulfate, diethanolamine laureth sulfate, lauric monoglyceride sodium sulfate, sodium lauryl sulfate, sodium laureth sulfate, potassium lauryl sulfate, potassium laureth sulfate, sodium lauryl sarcosinate, sodium lauroyl sarcosinate, lauryl sarcosine, cocoyl sarcosine, ammonium cocoyl sulfate, ammonium lauroyl sulfate, sodium cocoyl sulfate, sodium lauroyl sulfate, potassium cocoyl sulfate, potassium lauryl sulfate, triethanolamine lauryl sulfate, triethanolamine lauryl sulfate, monoethanolamine cocoyl sulfate, monoethanolamine lauryl sulfate, sodium tridecyl benzene sulfonate, sodium dodecyl benzene sulfonate, and combinations thereof.

Surfactant systems useful in the present invention may also comprise cationic surfactants. Cationic surfactants typically contain amino or quaternary ammonium hydrophilic moieties which are positively charged when dissolved in the aqueous composition of the present invention. Cationic surfactants among those useful herein are disclosed in the following documents: McCutcheon's, Detergents & Emulsifiers, (M.C. Publishing Co., North American edition 1989); Schwartz, et al., Surface Active Agents, Their Chemistry and Technology. New York: Interscience Publishers, 1949; U.S. Pat. Nos. 3,155,591; 3,929,678; 3,959,461; and 4,387,090.

Among the quaternary ammonium-containing cationic surfactant materials useful herein are those of the general formula:

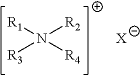

wherein R₁-R₄ are independently an aliphatic group of from about 1 to about 22 carbon atoms, or an aromatic, alkoxy, polyoxyalkylene, alkylamido, hydroxyalkyl, aryl or alkylaryl group having from about 12 to about 22 carbon atoms; and X is an anion selected from halogen, acetate, phosphate, nitrate and alkylsulfate radicals. The aliphatic groups may contain, in addition to carbon and hydrogen atoms, ether linkages, and other groups such as amino groups.

Other quaternary ammonium salts useful herein have the formula:

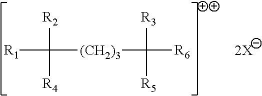

wherein R₁ is an aliphatic group having from about 16 to about 22 carbon atoms, R₂, R₃, R₄, R₅, and R₆ are selected from hydrogen and alkyl having from about 1 to about 4 carbon atoms, and X is an ion selected from halogen, acetate, phosphate, nitrate and alkyl sulfate radicals. Such quaternary ammonium salts include tallow propane diammonium dichloride.

Quaternary ammonium salts include monoalkyltrimethylammonium chlorides and dialkyldimethylammonium chlorides and trialkyl methyl ammonium chlorides, wherein at least one of the alkyl groups have from about 12 to about 22 carbon atoms and are derived from long-chain fatty acids, such as hydrogenated tallow fatty acid (tallow fatty acids yield quaternary compounds wherein the long chain alkyl groups are predominately from 16 to 18 carbon atoms). Examples of quaternary ammonium salts useful in the present invention include, but are not limited to, stearyl trimethyl ammonium chloride, ditallowedimethyl ammonium chloride, ditallowedimethyl ammonium methyl sulfate, dihexadecyl dimethyl ammonium chloride, di(hydrogenated tallow) dimethyl ammonium chloride, dioctadecyl dimethyl ammonium chloride, dieicosyl dimethyl ammonium chloride, didocosyl dimethyl ammonium chloride, di(hydrogenated tallow) dimethyl ammonium acetate, dihexadecyl dimethyl ammonium chloride, dihexadecyl dimethyl ammonium acetate, ditallow dipropyl ammonium phosphate, ditallow dimethyl ammonium nitrate, di(coconutalkyl) dimethyl ammonium chloride, and stearyl dimethyl benzyl ammonium chloride, ditallow dimethyl ammonium chloride, dicetyl dimethyl ammonium chloride, stearyl dimethyl benzyl ammonium chloride and cetyl trimethyl ammonium chloride are preferred quaternary ammonium salts useful herein.

In addition to the abovedescribed anionic and cationic surfactants, amphoteric surfactant components useful in the present composition include those known to be useful in personal cleansing compositions. Examples of amphoteric surfactants suitable for use in the composition herein are described in U.S. Pat. No. 5,104,646 (Bolich Jr., et al.) and U.S. Pat. No. 5,106,609 (Bolich Jr., et al.). Examples of amphoteric detersive surfactants which can be used in the compositions of the present invention are those which are broadly described as derivatives of aliphatic secondary and tertiary amines in which the aliphatic radical can be straight or branched chain and wherein one of the aliphatic substituents contains from about 8 to about 18 carbon atoms and one contains an anionic water solubilizing group, e.g., carboxy, sulfonate, sulfate, phosphate, or phosphonate.

Other amphoterics, sometimes classified as zwitterionics, such as betaines can also be used in the present invention. Such zwitterionics are considered as amphoterics in the present invention where the zwitterionic has an attached group that is anionic at the pH of the composition. Examples of betaines useful herein include the high alkyl betaines, such as. The sulfobetaines may be represented by coco dimethyl sulfopropyl betaine, stearyl dimethyl sulfopropyl betaine, lauryl dimethyl sulfoethyl betaine, lauryl bis-(2-hydroxyethyl) sulfopropyl betaine and the like; amidobetaines and amidosulfobetaines, wherein the RCONH(CH₂)₃ radical is attached to the nitrogen atom of the betaine are also useful in this invention.

Specifically, examples of amphoteric surfactants for use in the invention include: coco dimethyl carboxymethyl betaine, cocoamidopropyl betaine, cocobetaine, lauryl amidopropyl betaine, oleyl betaine, lauryl dimethyl carboxymethyl betaine, lauryl dimethyl alphacarboxyethyl betaine, cetyl dimethyl carboxymethyl betaine, lauryl bis-(2-hydroxyethyl)carboxymethyl betaine, stearyl bis-(2-hydroxypropyl)carboxymethyl betaine, oleyl dimethyl gamma-carboxypropyl betaine, and lauryl bis-(2-hydroxypropyl)-α-carboxyethyl betaine. Other examples of amphoteric surfactants are sodium 3-dodecyl-aminopropionate, sodium 3-dodecylaminopropane sulfonate, sodium lauroamphoacetate, N-alkyltaurines such as the one prepared by reacting dodecylamine with sodium isethionate according to the teaching of U.S. Pat. No. 2,658,072, N-higher alkyl aspartic acids such as those produced according to the teaching of U.S. Pat. No. 2,438,091, and the products sold under the trade name Miranol™ and described in U.S. Pat. No. 2,528,378.

Experience has shown that customary addition levels of the alcohol, acid, and/or surfactant are sufficient to produce the combination viscosity benefit with lightly- to moderately-crosslinked PVP. That is, the preferred addition level of each alcohol, acid, or surfactant is from 0.1% (w/w) to 25% (w/w), and more preferably ranges from 1% (w/w) to 10% (w/w) to induce the viscosity enhancement. One skilled in the art understands the necessary steps needed to evaluate the most preferred addition level for a particular formulation.

One or more alcohol(s), acid(s), and surfactant(s) may be used, as well as combinations thereof.

### Optional Ingredient: Conditioning Agents

Any known conditioning agent is useful in sun- and skin-care compositions of this invention, especially those conditioners that function to improve the cosmetic properties of the hair (i.e., softness, thickening, untangling, feel, and static electricity) and skin (i.e., moisturizing, anti-wrinkle, anti-cellulite, anti-blemish, firming, tightening, and bleaching). These conditioning agents may be in the form of a liquid, semi-solid, or solid form such as oils, waxes, or gums. Preferred conditioning agents include cationic polymers, cationic surfactants and cationic silicones.

Conditioning agents may be chosen from synthesis oils, mineral oils, vegetable oils, fluorinated or perfluorinated oils, natural or synthetic waxes, silicones, cationic polymers, proteins and hydrolyzed proteins, ceramide type compounds, cationic surfactants, fatty amines, fatty acids and their derivatives, as well as mixtures of these different compounds.

The synthesis oils include polyolefins, e.g., poly-α-olefins such as polybutenes, polyisobutenes and polydecenes. The polyolefins can be hydrogenated.

The mineral oils suitable for use in the compositions of the invention include hexadecane and oil of paraffin.

A list of suitable animal and vegetable oils comprises sunflower, corn, soy, avocado, jojoba, squash, raisin seed, sesame seed, walnut oils, fish oils, glycerol tricaprocaprylate, Purcellin oil or liquid jojoba, and blends thereof.

Suitable natural or synthetic oils include eucalyptus, lavender, vetiver, litsea cubeba, lemon, sandalwood, rosemary, chamomile, savory, nutmeg, cinnamon, hyssop, caraway, orange, geranium, cade, and bergamot.

Suitable natural and synthetic waxes include carnauba wax, candelila wax, alfa wax, paraffin wax, ozokerite wax, vegetable waxes such as olive wax, rice wax, hydrogenated jojoba wax, absolute flower waxes such as black currant flower wax, animal waxes such as bees wax, modified bees wax (cerabellina), marine waxes and polyolefin waxes such as polyethylene wax, and blends thereof.

The cationic polymers that may be used as a conditioning agent according to the invention are those known to improve the cosmetic properties of hair treated by detergent compositions. The expression “cationic polymer” as used herein, indicates any polymer containing cationic groups and/or ionizable groups in cationic groups. The cationic polymers used generally have a molecular weight the average number of which falls between about 500 Da and 5,000,000 Da and preferably between 1000 Da and 3,000,000 Da.

The preferred cationic polymers are chosen from among those containing units including primary, secondary, tertiary, and/or quaternary amine groups that may either form part of the main polymer chain or a side chain.

Useful cationic polymers include known polyamine, polyaminoamide, and quaternary polyammonium types of polymers, such as:
(1) Homopolymers and copolymers derived from acrylic or methacrylic esters or amides. The copolymers can contain one or more units derived from acrylamides, methacrylamides, diacetone acrylamides, acrylamides and methacrylamides, acrylic or methacrylic acids or their esters, vinyllactams such as vinyl pyrrolidone or vinyl caprolactam, and vinyl esters. Specific examples include: copolymers of acrylamide and dimethyl amino ethyl methacrylate quaternized with dimethyl sulfate or with an alkyl halide; copolymers of acrylamide and methacryloyl oxyethyl trimethyl ammonium chloride; the copolymer of acrylamide and methacryloyl oxyethyl trimethyl ammonium methosulfate; copolymers of vinyl pyrrolidone/dialkylaminoalkyl acrylate or methacrylate, optionally quaternized, such as the products sold under the name Gafquat® by International Specialty Products; the dimethyl amino ethyl methacrylate/vinyl caprolactam/vinyl pyrrolidone terpolymers, such as the product sold under the name Gaffix® VC 713 by International Specialty Products; the vinyl pyrrolidone/methacrylamidopropyl dimethylamine copolymer, marketed under the name Styleze® CC 10 by International Specialty Products; the vinyl pyrrolidone/quaternized dimethyl amino propyl methacrylamide copolymers such as the product sold under the name Gafquat® HS 100 by International Specialty Products (Wayne, N.J.), and the terpolymer of N-vinyl-2-pyrrolidone, dimethylaminopropyl methacrylamide, and methacryloylaminopropyl lauryl dimethylammonium chloride, sold under the name Styleze® W, also by International Specialty Products.
(2) Derivatives of cellulose ethers containing quaternary ammonium groups, such as hydroxy ethyl cellulose quaternary ammonium that has reacted with an epoxide substituted by a trimethyl ammonium group.
(3) Derivatives of cationic cellulose such as cellulose copolymers or derivatives of cellulose grafted with a hydrosoluble quaternary ammonium monomer, as described in U.S. Pat. No. 4,131,576, such as the hydroxy alkyl cellulose, and the hydroxymethyl-, hydroxyethyl- or hydroxypropyl-cellulose grafted with a salt of methacryloyl ethyl trimethyl ammonium, methacrylamidopropyl trimethyl ammonium, or dimethyl diallyl ammonium.
(4) Cationic polysaccharides such as described in U.S. Pat. Nos. 3,589,578 and 4,031,307, guar gums containing cationic trialkyl ammonium groups and guar gums modified by a salt, e.g., chloride of 2,3-epoxy propyl trimethyl ammonium.
(5) Polymers composed of piperazinyl units and alkylene or hydroxy alkylene divalent radicals with straight or branched chains, possibly interrupted by atoms of oxygen, sulfur, nitrogen, or by aromatic or heterocyclic cycles, as well as the products of the oxidation and/or quaternization of such polymers.
(6) Water-soluble polyamino amides prepared by polycondensation of an acid compound with a polyamine. These polyamino amides may be reticulated.
(7) Derivatives of polyamino amides resulting from the condensation of polyalcoylene polyamines with polycarboxylic acids followed by alcoylation by bi-functional agents.
(8) Polymers obtained by reaction of a polyalkylene polyamine containing two primary amine groups and at least one secondary amine group with a dioxycarboxylic acid chosen from among diglycolic acid and saturated dicarboxylic aliphatic acids having 3 to 8 atoms of carbon. Such polymers are described in U.S. Pat. Nos. 3,227,615 and 2,961,347.

(9) Cyclopolymers of alkyl dialyl amine or dialkyl diallyl ammonium such as the homopolymer of dimethyl diallyl ammonium chloride and copolymers of diallyl dimethyl ammonium chloride and acrylamide.
(10) Quaternary diammonium polymers such as hexadimethrine chloride.
(11) Quaternary polyammonium polymers, including, for example, Mirapol® A 15, Mirapol® AD1, Mirapol® AZ1, and Mirapol® 175 products sold by Miranol.
(12) Quaternary polymers of vinyl pyrrolidone and vinyl imidazole such as the products sold under the names Luviquat® FC 905, FC 550, and FC 370 by BASF Corporation.
(13) Quaternary polyamines.
(14) Reticulated polymers known in the art.

Other cationic polymers that may be used within the context of the invention are cationic proteins or hydrolyzed cationic proteins, polyalkyleneimines such as polyethyleneimines, polymers containing vinyl pyridine or vinyl pyridinium units, condensates of polyamines and epichlorhydrins, quaternary polyurethanes, and derivatives of chitin.

Preferred cationic polymers are derivatives of quaternary cellulose ethers, the homopolymers and copolymers of dimethyl diallyl ammonium chloride, quaternary polymers of vinyl pyrrolidone and vinyl imidazole, and mixtures thereof.

The conditioning agent can be any silicone known by those skilled in the art to be useful as a conditioning agent. The silicones suitable for use according to the invention include polyorganosiloxanes that are insoluble in the composition. The silicones may be present in the form of oils, waxes, resins, or gums. They may be volatile or non-volatile. The silicones can be selected from polyalkyl siloxanes, polyaryl siloxanes, polyalkyl aryl siloxanes, silicone gums and resins, and polyorgano siloxanes modified by organofunctional groups, and mixtures thereof.

Suitable polyalkyl siloxanes include polydimethyl siloxanes with terminal trimethyl silyl groups or terminal dimethyl silanol groups (dimethiconol) and polyalkyl (C1-C20) siloxanes.

Suitable polyalkyl aryl siloxanes include polydimethyl methyl phenyl siloxanes and polydimethyl diphenyl siloxanes, linear or branched.

The silicone gums suitable for use herein include polydiorganosiloxanes preferably having a number-average molecular weight between 200,000 Da and 1,000,000, Da used alone or mixed with a solvent. Examples include polymethyl siloxane, polydimethyl siloxane/methyl vinyl siloxane gums, polydimethyl siloxane/diphenyl siloxane, polydimethyl siloxane/phenyl methyl siloxane and polydimethyl siloxane/diphenyl siloxane/methyl vinyl siloxane.

Suitable silicone resins include silicones with a dimethyl/trimethyl siloxane structure and resins of the trimethyl siloxysilicate type.

The organo-modified silicones suitable for use in the invention include silicones such as those previously defined and containing one or more organofunctional groups attached by means of a hydrocarbon radical and grafted siliconated polymers. Particularly preferred are amino functional silicones.

The silicones may be used in the form of emulsions, nano-emulsions, or micro-emulsions.

The conditioning agent can be a protein or hydrolyzed cationic or non-cationic protein. Examples of these compounds include hydrolyzed collagens having triethyl ammonium groups, hydrolyzed collagens having trimethyl ammonium and trimethyl stearyl ammonium chloride groups, hydrolyzed animal proteins having trimethyl benzyl ammonium groups (benzyltrimonium hydrolyzed animal protein), hydrolyzed proteins having groups of quaternary ammonium on the polypeptide chain, including at least one C1-C18 alkyl.

Hydrolyzed proteins include Croquat L, in which the quaternary ammonium groups include a C12 alkyl group, Croquat M, in which the quaternary ammonium groups include C10-C18 alkyl groups, Croquat S in which the quaternary ammonium groups include a C18 alkyl group and Crotein Q in which the quaternary ammonium groups include at least one C1-C18 alkyl group. These products are sold by Croda.

The conditioning agent can comprise quaternized vegetable proteins such as wheat, corn, or soy proteins such as cocodimonium hydrolyzed wheat protein, laurdimonium hydrolyzed wheat protein and steardimonium hydrolyzed wheat protein, 2-N-stearoyl amino-octadecane-1,3-diol, 2-N-behenoyl amino-octadecane-1,3-diol, 2-N-[2-hydroxy-palmitoyl]-amino-octadecane-1,3-diol, 2-N-stearoyl amino-octadecane-1,3,4-triol, N-stearoyl phytosphingosine, 2-N-palmitoyl amino-hexadecane-1,3-diol, bis-(N-hydroxy ethyl N-cetyl)malonamide, N-(2-hydroxy ethyl)-N-(3-cetoxyl-2-hydroxy propyl)amide of cetylic acid, N-docosanoyl N-methyl-D-glucamine and mixtures of such compounds.

The conditioning agent can be a cationic surfactant such as a salt of a primary, secondary, or tertiary fatty amine, optionally polyoxyalkylenated, a quaternary ammonium salt, a derivative of imadazoline, or an amine oxide. Suitable examples include mono-, di-, or tri-alkyl quaternary ammonium compounds with a counterion such as a chloride, methosulfate, tosylate, etc. including, but not limited to, cetrimonium chloride, dicetyldimonium chloride, behentrimonium methosulfate, and the like. The presence of a quaternary ammonium compound in conjunction with the polymer described above reduces static and enhances combing of hair in the dry state. The polymer also enhances the deposition of the quaternary ammonium compound onto the hair substrate thus enhancing the conditioning effect of hair.

The conditioning agent can be any fatty amine known to be useful as a conditioning agent; e.g. dodecyl, cetyl or stearyl amines, such as stearamidopropyl dimethylamine.

The conditioning agent can be a fatty acid or derivatives thereof known to be useful as conditioning agents. Suitable fatty acids include myristic acid, palmitic acid, stearic acid, behenic acid, oleic acid, linoleic acid, and isostearic acid. The derivatives of fatty acids include carboxylic ester acids including mono-, di-, tri- and tetra-carboxylic acids.

The conditioning agent can be a fluorinated or perfluorinated oil. The fluoridated oils may also be fluorocarbons such as fluoramines, e.g., perfluorotributylamine, fluoridated hydrocarbons, such as perfluorodecahydronaphthalene, fluoroesters, and fluoroethers.

Of course, mixtures of two or more conditioning agents can be used.

The conditioning agent or agents can be present in an amount of 0.001% to 20%, preferably from 0.01% to 10%, and even more preferably from 0.1% to 3% by weight based on the total weight of the final composition.

The antioxidants or antiradical agents can be selected from phenols such as BHA (tert-butyl-4-hydroxy anisole), BHT (2,6-di-tert-butyl-p-cresol), TBHQ (tert-butyl hydroquinone), polyphenols such as proanthocyanodic oligomers, flavonoids, hindered amines such as tetra amino piperidine, erythorbic acid, polyamines such as spermine, cysteine, glutathione, superoxide dismutase, and lactoferrin.

The vitamins can be selected from ascorbic acid (vitamin C), vitamin E, vitamin E acetate, vitamin E phosphate, B vitamins such as B3 and B5, niacin, vitamin A, and derivatives thereof. The provitamins can be selected from panthenol and retinol.

The protecting agent can be present in an amount 0.001% to 20% by weight, preferably from 0.01% to 10% by weight, and more preferably 0.1 to 5% by weight of the total weight of the final composition.

### Optional Ingredients: Viscosity or Thickening Agents

Additional thickeners or viscosity increasing agents may be included in the composition of the invention, such as: Acetamide MEA; acrylamide/ethalkonium chloride acrylate copolymer; acrylamide/ethyltrimonium chloride acrylate/ethalkonium chloride acrylate copolymer; acrylamides copolymer; acrylamide/sodium acrylate copolymer; acrylamide/sodium acryloyldimethyltaurate copolymer; acrylates/acetoacetoxyethyl methacrylate copolymer; acrylates/beheneth-25 methacrylate copolymer; acrylates/C10-C30 alkyl acrylate crosspolymer; acrylates/ceteth-20 itaconate copolymer; acrylates/ceteth-20 methacrylate copolymer; acrylates/laureth-25 methacrylate copolymer; acrylates/palmeth-25 acrylate copolymer; acrylates/palmeth-25 itaconate copolymer; acrylates/steareth-50 acrylate copolymer; acrylates/steareth-20 itaconate copolymer; acrylates/steareth-20 methacrylate copolymer; acrylates/stearyl methacrylate copolymer; acrylates/vinyl isodecanoate crosspolymer; acrylic acid/acrylonitrogens copolymer; adipic acid/methyl DEA crosspolymer; agar; agarose; alcaligenes polysaccharides; algin; alginic acid; almondamide DEA; almondamidopropyl betaine; aluminum/magnesium hydroxide stearate; ammonium acrylates/acrylonitrogens copolymer; ammonium acrylates copolymer; ammonium acryloyldimethyltaurate/vinyl formamide copolymer; ammonium acryloyldimethyltaurate/VP copolymer; ammonium alginate; ammonium chloride; ammonium polyacryloyldimethyl taurate; ammonium sulfate; amylopectin; apricotamide DEA; apricotamidopropyl betaine; arachidyl alcohol; arachidyl glycol; arachis hypogaea (peanut) flour; ascorbyl methylsilanol pectinate; astragalus gummifer gum; attapulgite; avena sativa (oat) kernel flour; avocadamide DEA; avocadamidopropyl betaine; azelamide MEA; babas suamide DEA; babassuamide MEA; babassuamidopropyl betaine; behenamide DEA; behenamide MEA; behenamidopropyl betaine; behenyl betaine; bentonite; butoxy chitosan; caesalpinia spinosa gum; calcium alginate; calcium carboxymethyl cellulose; calcium carrageenan; calcium chloride; calcium potassium carbomer; calcium starch octenylsuccinate; C20-40 alkyl stearate; canolamidopropyl betaine; capramide DEA; capryl/capramidopropyl betaine; carbomer; carboxybutyl chitosan; carboxymethyl cellulose acetate butyrate; carboxymethyl chitin; carboxymethyl chitosan; carboxymethyl dextran; carboxymethyl hydroxyethylcellulose; carboxymethyl hydroxypropyl guar; carnitine; cellulose acetate propionate carboxylate; cellulose gum; ceratonia siliqua gum; cetearyl alcohol; cetyl alcohol; cetyl babassuate; cetyl betaine; cetyl glycol; cetyl hydroxyethylcellulose; chimyl alcohol; cholesterol/HDI/pullulan copolymer; cholesteryl hexyl dicarbamate pullulan; citrus aurantium dulcis (orange) peel extract; cocamide DEA; cocamide MEA; cocamide MIPA; cocamidoethyl betaine; cocamidopropyl betaine; cocamidopropyl hydroxysultaine; coco-betaine; coco-hydroxysultaine; coconut alcohol; coco/oleamidopropyl betaine; coco-Sultaine; cocoyl sarcosinamide DEA; cornamide/cocamide DEA; cornamide DEA; croscarmellose; crosslinked bacillus/glucose/sodium glutamate ferment; cyamopsis tetragonoloba (guar) gum; decyl alcohol; decyl betaine; dehydroxanthan gum; dextrin; dibenzylidene sorbitol; diethanolaminooleamide DEA; diglycol/CHDM/isophthalates/S IP copolymer; dihydroabietyl behenate; dihydrogenated tallow benzylmonium hectorite; dihydroxyaluminum aminoacetate; dimethicone/PEG-10 crosspolymer; dimethicone/PEG-15 crosspolymer; dimethicone propyl PG-betaine; dimethylacrylamide/acrylic acid/polystyrene ethyl methacrylate copolymer; dimethylacrylamide/sodium acryloyldimethyltaurate crosspolymer; disteareth-100 IPDI; DMAPA acrylates/acrylic acid/acrylonitrogens copolymer; erucamidopropyl hydroxysultaine; ethylene/sodium acrylate copolymer; gelatin; gellan gum; glyceryl alginate; glycine soja (soybean) flour; guar hydroxypropyltrimonium chloride; hectorite; hyaluronic acid; hydrated silica; hydrogenated potato starch; hydrogenated tallow; hydrogenated tallowamide DEA; hydrogenated tallow betaine; hydroxybutyl methylcellulose; hydroxyethyl acrylate/sodium acryloyldimethyl taurate copolymer; hydroxyethylcellulose; hydroxyethyl chitosan; hydroxyethyl ethylcellulose; hydroxyethyl stearamide-MIPA; hydroxylauryl/hydroxymyristyl betaine; hydroxypropylcellulose; hydroxypropyl chitosan; hydroxypropyl ethylenediamine carbomer; hydroxypropyl guar; hydroxypropyl methylcellulose; hydroxypropyl methylcellulose stearoxy ether; hydroxypropyl starch; hydroxypropyl starch phosphate; hydroxypropyl xanthan gum; hydroxystearamide MEA; isobutylene/sodium maleate copolymer; isostearamide DEA; isostearamide MEA; isostearamide mIPA; isostearamidopropyl betaine; lactamide MEA; lanolinamide DEA; lauramide DEA; lauramide MEA; lauramide MIPA; lauramide/myristamide DEA; lauramidopropyl betaine; lauramidopropyl hydroxysultaine; laurimino bispropanediol; lauryl alcohol; lauryl betaine; lauryl hydroxysultaine; lauryl/myristyl glycol hydroxypropyl ether; lauryl sultaine; lecithinamide DEA; linoleamide DEA; linoleamide MEA; linoleamide MIPA; lithium magnesium silicate; lithium magnesium sodium silicate; macrocystis pyrifera (kelp); magnesium alginate; magnesium/aluminum/hydroxide/carbonate; magnesium aluminum silicate; magnesium silicate; magnesium trisilicate; methoxy PEG-22/dodecyl glycol copolymer; methylcellulose; methyl ethylcellulose; methyl hydroxyethylcellulose; microcrystalline cellulose; milkamidopropyl betaine; minkamide DEA; minkamidopropyl betaine; MIPA-myristate; montmorillonite; Moroccan lava clay; myristamide DEA; myristamide MEA; myristamide MIPA; myristamidopropyl betaine; myristamidopropyl hydroxysultaine; myristyl alcohol; myristyl betaine; natto gum; nonoxynyl hydroxyethylcellulose; oatamide MEA; oatamidopropyl betaine; octacosanyl glycol isostearate; octadecene/MA copolymer; oleamide DEA; oleamide MEA; oleamide MIPA; oleamidopropyl betaine; oleamidopropyl hydroxysultaine; oleyl betaine; olivamide DEA; olivamidopropyl betaine; oliveamide MEA; palmamide DEA; palmamide MEA; palmamide MIPA; palmamidopropyl betaine; palmitamide DEA; palmitamide MEA; palmitamidopropyl betaine; palm kernel alcohol; palm kernelamide DEA; palm kernelamide MEA; palm kernelamide MIPA; palm kernelamidopropyl betaine; peanutamide MEA; peanutamide MIPA; pectin; PEG-800; PEG-crosspolymer; PEG-150/decyl alcohol/SMDI copolymer; PEG-175 diisostearate; PEG-190 distearate; PEG-15 glyceryl tristearate; PEG-140 glyceryl tristearate; PEG-240/HDI copolymer bis-decyltetradeceth-20 ether; PEG-100/IPDI copolymer; PEG-180/laureth-50/TMMG copolymer; PEG-10/lauryl dimethicone crosspolymer; PEG-15/lauryl dimethicone crosspolymer; PEG-2M; PEG-5M; PEG-7M; PEG-9M; PEG-14M; PEG-20M; PEG-23M; PEG-25M; PEG-45M; PEG-65M; PEG-90M; PEG-115M; PEG-160M; PEG-180M; PEG-120 methyl glucose trioleate; PEG-180/octoxynol-40/TMMG copolymer; PEG-150 pentaerythrityl tetrastearate; PEG-4 rapeseedamide; PEG-150/stearyl alcohol/SMDI copolymer; phaseolus angularis seed powder; polianthes tuberosa extract; polyacrylate-3; polyacrylic acid; polycyclopentadiene; polyether-1; polyethylene/isopropyl maleate/MA copolyol; polyglyceryl-3 disiloxane dimethicone; polyglyceryl-3 polydimethylsiloxyethyl dimethicone; polymethacrylic acid; polyquaternium-52; polyvinyl alcohol; potassium alginate; potassium aluminum polyacrylate; potassium carbomer; potassium carrageenan; potassium chloride; potassium palmate; potassium polyacrylate; potassium sulfate; potato starch modified; PPG-2 cocamide; PPG-1 hydroxyethyl caprylamide; PPG-2 hydroxyethyl cocamide; PPG-2 hydroxyethyl coco/isostearamide; PPG-3 hydroxyethyl soyamide; PPG-14 laureth-60 hexyl dicarbamate; PPG-14 laureth-60 isophoryl dicarbamate; PPG-14 palmeth-60 hexyl dicarbamate; propylene glycol alginate; PVP/decene copolymer; PVP montmorillonite; pyrus cydonia seed; pyrus malus (apple) fiber; rhizobian gum; ricebranamide DEA; ricinoleamide DEA; ricinoleamide MEA; ricinoleamide MIPA; ricinoleamidopropyl betaine; ricinoleic acid/adipic acid/AEEA copolymer; rosa multiflora flower wax; sclerotium gum; sesamide DEA; sesamidopropyl betaine; sodium acrylate/acryloyldimethyl taurate copolymer; sodium acrylates/acrolein copolymer; sodium acrylates/acrylonitrogens copolymer; sodium acrylates copolymer; sodium acrylates crosspolymer; sodium acrylate/sodium acrylamidomethylpropane sulfonate copolymer; sodium acrylates/vinyl isodecanoate crosspolymer; sodium acrylate/vinyl alcohol copolymer; sodium carbomer; sodium carboxymethyl chitin; sodium carboxymethyl dextran; sodium carboxymethyl beta-glucan; sodium carboxymethyl starch; sodium carrageenan; sodium cellulose sulfate; sodium chloride; sodium cyclodextrin sulfate; sodium hydroxypropyl starch phosphate; sodium isooctylene/MA copolymer; sodium magnesium fluorosilicate; sodium oleate; sodium palmitate; sodium palm kernelate; sodium polyacrylate; sodium polyacrylate starch; sodium polyacryloyldimethyl taurate; sodium polygamma-glutamate; sodium polymethacrylate; sodium polystyrene sulfonate; sodium silicoaluminate; sodium starch octenylsuccinate; sodium stearate; sodium stearoxy PG-hydroxyethylcellulose sulfonate; sodium styrene/acrylates copolymer; sodium sulfate; sodium tallowate; sodium tauride acrylates/acrylic acid/acrylonitrogens copolymer; sodium tocopheryl phosphate; solanum tuberosum (potato) starch; soyamide DEA; soyamidopropyl betaine; starch/acrylates/acrylamide copolymer; starch hydroxypropyltrimonium chloride; stearamide AMP; stearamide DEA; stearamide DEA-distearate; stearamide DIBA-stearate; stearamide MEA; stearamide MEA-stearate; stearamide MIPA; stearamidopropyl betaine; steareth-60 cetyl ether; steareth-100/PEG-136/HDI copolymer; stearyl alcohol; stearyl betaine; sterculia urens gum; synthetic fluorphlogopite; tallamide DEA; tallow alcohol; tallowamide DEA; tallowamide MEA; tallowamidopropyl betaine; tallowamidopropyl hydroxysultaine; tallowamine oxide; tallow betaine; tallow dihydroxyethyl betaine; tamarindus indica seed gum; tapioca starch; TEA-alginate; TEA-carbomer; TEA-hydrochloride; trideceth-2 carboxamide MEA; tridecyl alcohol; triethylene glycol dibenzoate; trimethyl pentanol hydroxyethyl ether; triticum vulgare (wheat) germ powder; triticum vulgare (wheat) kernel flour; triticum vulgare (wheat) starch; tromethamine acrylates/acrylonitrogens copolymer; tromethamine magnesium aluminum silicate; undecyl alcohol; undecylenamide DEA; undecylenamide MEA; undecylenamidopropyl betaine; welan gum; wheat germamide DEA; wheat germamidopropyl betaine; xanthan gum; yeast beta-glucan; yeast polysaccharides and zea mays (corn) starch.

### Product Forms

The sun- and skin-care compositions of this invention lend themselves to a large number of product forms. For example, depending in part on the addition level of the lightly- to moderately-crosslinked PVP (and other ingredients, like viscosity synergists), they may assume a consistency ranging from liquid to semiliquid forms (e.g., milks, creams), to thicker forms like gels, creams, pastes, and even solid- and wax-like forms.

Optionally, the compositions described herein may be packaged in aerosol form and may be provided in the form of a mousse, spray, or mist. It may be advantageous to utilize known propellants (e.g., hydrofluorinated compounds dichlorodifluoromethane, difluoroethane, dimethyl ether, isobutene, n-butane, propane, trichlorofluoromethane) to aide in their delivery.

When the sun-care composition contains a high level of alcohol, such as ethanol, they may tend to evaporate more quickly, spread more easily, feel non-greasy, and/or exhibit less whitening than water-only based compositions.

While skin-care compositions are directed to application on the skin, sun-care compositions protect the wearer from the sun, and may be applied to the skin, hair, or lips.

Suitable end-use products that can be created from the sun-care compositions include, without limitation: lotions, sport lotions (i.e., being sweatproof, waterproof, resistant to running, having added UV absorbers and/or antioxidants), baby and sensitive skin products (i.e., having low irritation to the eyes and/or skin, and/or being free of fragrances and/or dyes), daily wear, lip care, continuous sprays, hair lotions, leave-on hair creams and styling aides, and hair sprays.

The invention also lends itself to skin-care products that include, without limitation: creams, gels, lotions, ointments, and sprays for diaper rash, insect bites, contact dermatosis, and sunburn.

A non-limiting embodiment of the invention now will be provided, with the understanding that one skilled in the art can develop other examples embraced by the invention.

### EXAMPLES

### Example 1

A sun-care composition of the invention was prepared containing ethanol, titanium dioxide, and zinc oxide (Table 1). Although the sun-care composition did not contain any water, both inorganic UV absorbers remained suspended throughout the formula. Ingredient incompatibilities, such as inorganic UV absorber settling, phase separation, agglomeration, and color change, were not noted.

The above sun-care composition exhibited excellent spreadability, dried quickly, and did not impart a whitening effect. During application it was found not to pill (i.e., form into small balls).

TABLE 1
The formula for the sun-care composition of Example 1

| | | | addition |
|---|---|---|---|
| ingredient | | | level |
| INCI name | brand name | supplier | (% w/w) |
| Phase A: | | | |
| ethanol | | | 64.75 |
| lightly-to moderately- | FlexiThix ™ | ISP | 3.00 |
| crosslinked PVP | | | |
| Phase B: | | | |
| C12-C15 alkyl lactates | Ceraphyl ® 41 | ISP | 5.00 |
| coated fine particle | Tioveil ™ CM* | Croda | 15.25 |
| titanium dioxide in | | | |
| cyclomethicone pentamer | | | |
| cyclopentasiloxane (and) | SiTec ™ RE100 | ISP | 5.00 |
| dimethicone/vinyltrimethyl | | | |
| siloxysilicate crosspolymer | | | |
| hydrophilic derivative | Cera Bellina | Koster | 1.00 |
| of natural beeswax in | | | |
| which the free fatty acids | | | |
| have been converted | | | |
| to polyglycerols esters | | | |
| isocetyl seteroyl stearate | Ceraphyl ® 791 | ISP | 3.00 |
| Phase C: | | | |
| very fine, amorphous | Zoco 112 | Zochem Inc. | 3.00 |
| zinc oxide | | | |
| | | total: | 100.00 |

*Tioveil ™ CM contains 33% active TiO₂

What is claimed is:

1. A personal care composition comprising: (A) at least one metal oxide, (B) lightly- to moderately-crosslinked PVP, and (C) at least one vehicle selected from the group consisting of: alcohols, esters, oils, glycols, and combinations thereof.

2. The personal care compositions according to claim 1 that is a sun-care or skin-care composition.

3. The personal care composition according to claim 1 wherein said metal oxide is selected from the group consisting of: titanium dioxides, zinc oxides, iron oxides, and combinations thereof.

4. The personal care composition according to claim 1 wherein said metal oxide is selected from the group consisting of: coated metal oxides, uncoated metal oxides, and combinations thereof.

5. The personal care composition according to claim 1 wherein said metal oxide is present in an amount from 0.1% to 40% based on the total weight of said personal care composition.

6. The personal care composition according to claim 1 wherein said lightly- to moderately-crosslinked PVP exhibits a gel volume in deionized water from 15 mL/g to 300 mL/g.

7. The personal care composition according to claim 1 wherein said vehicle is present in an amount from 5% to 95% based on the total weight of said personal care composition.

8. The personal care composition according to claim 1 wherein said alcohol is selected from the group consisting of: ethanol, 1-propanol, 2-propanol, and combinations thereof.

9. The personal care composition according to claim 1 wherein said glycol is selected from the group consisting of: propylene glycol, butylene glycol, diethylene glycol, dipropylene glycol, polyethylene glycol, glycerin, and combinations thereof.

10. The personal care composition according to claim 1 wherein said oil is selected from the group consisting of: synthetic oils, silicone oils, mineral oils, vegetable oils, and combinations thereof.

11. The personal care composition according to claim 10 wherein said synthetic oil is selected from the group consisting of: silicones, polybutenes, polyisobutenes, polydecenes, and combinations thereof.

12. The personal care composition according to claim 10 wherein said mineral oil is selected from the group consisting of: hexadecane, oil of paraffin, and combinations thereof.

13. The personal care combination according to claim 10 wherein said vegetable oil is selected from the group consisting of: sunflower oil, corn oil, soy oil, avocado oil, jojoba oil, squash oil, raisin seed oil, sesame seed oil, walnut oils oil, glycerol tricaprocaprylate, Purcellin oil, jojoba oil, eucalyptus oil, lavender oil, vetiver oil, litsea cubeba oil, lemon oil, sandalwood oil, rosemary oil, chamomile oil, savory oil, nutmeg oil, cinnamon oil, hyssop oil, caraway oil, orange oil, geranium oil, cade oil, oil of bergamot, and combinations thereof.

14. The personal care composition according to claim 1 wherein said ester is selected from the group consisting of: butyl ester of PVM/MA copolymer, cetyl ethylhexanoate, decyl oleate, diisopropyl adipate, diisopropyl dimer dilinoleate, diisostearyl maleate, dioctyl maleate, ethylhexyl palmitate, 2-ethylhexyl-12-hydroxystearate, ethyl ester of PVM/MA copolymer, isocetyl stearate, isocetyl stearoyl stearate, isodecyl neopentanoate, isodecyl oleate, isopropyl ester of PVM/MA copolymer, isostearyl neopentanoate, myristyl laurate, myristyl myristate, neopentyl glycol diethylhexanoate, octyldodecyl stearate, phenethyl benzoate, triisostearyoyl polyglyceryl-3 dimer dilinoleate, and combinations thereof.

15. The personal care composition according to claim 1 that further comprises water.

16. The personal care composition according to claim 15 wherein said water is present in an amount up to 15% based on the total weight of said personal care composition.

17. The personal care composition according to claim 1 that further comprises at least one UV absorber.

18. The personal care composition according to claim 17 wherein said additional UV absorber is selected from the group consisting of: octocrylene, cinoxate, 3-benzylidene camphor, 4-methylbenzylidene camphor, lisadimate, benzophenone-1, benzophenone-3; benzophenone-4; benzophenone-5; benzophenone-6, benzophenone-8, benzophenone-9, bisdisulizole disodium, bemotrizinol, bisoctrizole, camphor benzalkonium methosulfate, DEA methoxycinnamate, diethylaminohydroxybenzoylhexyl benzoate, iscotrizinol, digalloyl trioleate, diisopropyl methylcinnamate, dimethoxyphenyl-[1-(3,4)]-4,4-dimethyl 1,3-pentanedione, drometrizole, drometrizole trisiloxane, roxadimate, ethylhexyl-p-methoxycinnamate, ethylhexyl triazone, ferulic acid, glyceryl ethylhexanoate dimethoxycinnamate, homosalate, isoamyl-p-methoxycinnamate, lawsone with dihydroxyacetone, meradimate, padimate 0, octyl salicylate, PABA, PEG-25 PABA, padimate A, ensulizole, polyacrylamido methylbenzylidene camphor, polysilicone-15, encamsule, trolamine salicylate, titanium dioxide, zinc oxide, and combinations thereof.

19. The personal care composition according to claim 1 that further comprises at least one ingredient selected from the group consisting of: active ingredients, emollients, liquid carriers, waxes, conditioners, surfactants, emulsifiers, rheology modifiers, lubricants, diluents, humectants, anti-oxidants, preservatives, antibiotics, viscosity synergists, clarity syngergists, and combinations thereof.

20. The personal care composition according to claim 1 that is a: a lotion sunscreen, a cream sunscreen, a gel sunscreen, a sprayable sunscreen, a diaper rash cream, a diaper rash ointment, a diaper rash lotion, a diaper rash gel, an anti-pruritic lotion, and anti-pruritic cream, an anti-pruritic gel, a moisturizer, skin cream, eyelid cream, ointment, liniment, anti-aging cream, lip moisturizer, lipstick, lip gloss, hair spray, hair finishing spray, mousse, hair styling gel, hair styling cream, or hair styling wax.

21. The personal care composition according to claim 1 that is smooth and non-pilling when applied to the skin or hair.
